(19) United States
(12) Patent Application Publication
(10) Pub. No.: US 2023/0341427 A1
(43) Pub. Date: Oct. 26, 2023

(54) SYSTEMS AND METHODS OF SPECIMEN CONTAINER LABELING AND TRACKING

(71) Applicant: Board of Regents, The University of Texas System, Austin, TX (US)

(72) Inventors: Aisen C. Chacin, Galveston, TX (US); Christopher J. Zahner, Galveston, TX (US)

(21) Appl. No.: 18/303,120

(22) Filed: Apr. 19, 2023

Related U.S. Application Data
(60) Provisional application No. 63/332,879, filed on Apr. 20, 2022.

(51) Int. Cl. G01N 35/00; B01L 3/00

(52) U.S. Cl. CPC G01N 35/00732; B01L 3/545; G01N 2035/00752; B01L 2300/021; G16H 10/60

(57) ABSTRACT

The present disclosure presents systems and methods of specimen container labeling and tracking. One such method comprises linking a pair of identification numbers with one another within a specimen test order in an electronic medical record system in advance of the pair of identification numbers being printed on a double sided container label; receiving a first machine readable identifier on a first side of the label before a specimen is collected; transmitting a first identification number represented by a scanned first machine readable identifier to the record system; receiving a second machine readable identifier on a second side of the label after the specimen is collected; transmitting a second identification number represented by a scanned second machine readable identifier to the record system; and authorizing the specimen to be tested after verifying that the second identification number is associated with the specimen test order and/or the first machine readable identifier.

### CROSS-REFERENCE TO RELATED APPLICATION

This application claims priority to co-pending U.S. provisional application entitled, “Systems and Methods of Specimen Container Labeling and Tracking,” having Ser. No. 63/332,879, filed Ap. 20, 2022, which is entirely incorporated herein by reference.

### BACKGROUND

Currently specimen handling is challenging. As a result of the hurried pace, steps in the handling of specimens get skipped or are done out of order which can result in the wrong labeling of specimens. In a clinical setting, this mistake can result in fatal errors.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 illustrates the pairing of identification numbers (identifiers) for an exemplary double sided label in accordance with various embodiments of the present disclosure.

FIG. 2 shows an exemplary method of specimen container labeling in accordance with various embodiments of the present disclosure.

FIG. 3 shows an exemplary flow process for interfacing with an electronic medical record system as part of specimen container labeling in accordance with various embodiments of the present disclosure.

FIGS. 4-11 shows exemplary interface screens to an electronic medical record system as part of specimen container labeling in accordance with various embodiments of the present disclosure.

FIG. 12 shows an exemplary workflow process for labeling and collecting a specimen in accordance with various embodiments of the present disclosure.

FIG. 13 depicts a schematic block diagram of a computing device that can be used to implement various embodiments of the present disclosure.

### DETAILED DESCRIPTION

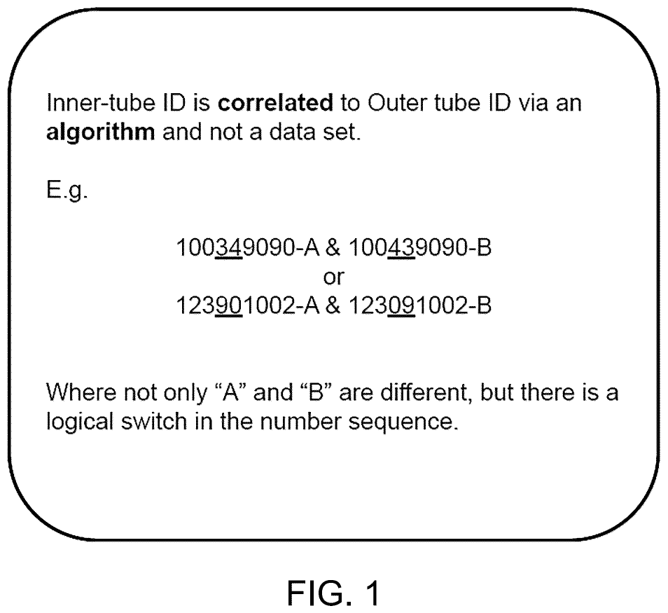

Improved systems and methods for labeling, handling, and tracking the sequence and timing of events before and after specimens are collected are presented within the present disclosure. An exemplary method utilizes a double sided label that is unique to each specimen tube or container, such that the double sided label has a barcode or other type of identifier on each side of the label. In accordance with the present disclosure, each of the bar codes/identifiers represents an identification number such that a pair of identification numbers is associated with the double sided label and the specimen container to which the label is attached. In various embodiments, the pair of identification numbers are linked or associated with one another within an electronic medical record database system in advance of being applied or adhered to a specimen tube or container (or other type of vessel for holding a specimen in accordance with various embodiments of the present disclosure). The linked or associated identification numbers prevent a user from decoding and manually entering the identification number into the computer system, thus the relationship between the pair of identification numbers can only be determined by the computer system. In various embodiments, the pair of identification numbers are different but related numbers in that a portion of each of the numbers (e.g., beginning, middle, or end digits of the number) may be the same and a different portion of each of the numbers (e.g., digits within a beginning, middle, or end portion) may be different, as demonstrated by FIG. 1.

One emphasis of the disclosure is the importance on the tracking the sequence and timing of events which cannot be currently done effectively. The double-sided label is an identifier for the container itself. Accordingly, the double-sided ID is adhered to the container prior to the bedside environment. Thus, the double-sided ID is not typically intended to be printed by a nurse at the bedside environment. For reference, in current practice, there are 2 labels to be scanned: the order label and the patient wristband (or other identifier worn by the subject, whether a person or other type of mammal). The present disclosure offers a third label that is affixed to the clear container either by the manufacturer, materials management, or the lab. Otherwise, this could create another opportunity to circumvent the prescribed order of events by scanning an unattached label after collecting the specimen.

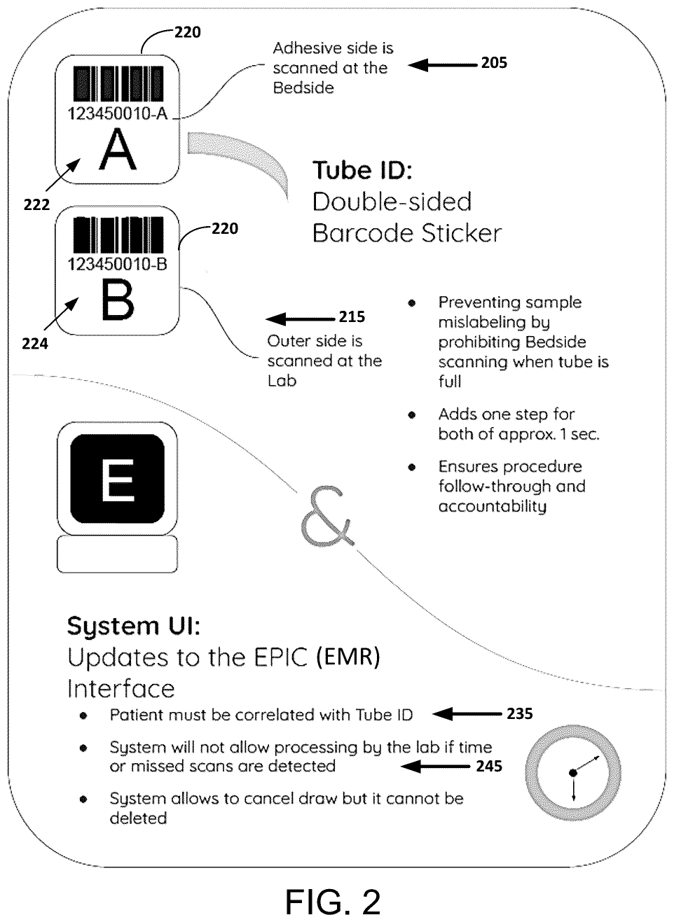

In an exemplary method, as illustrated in FIG. 2, a specimen collector (e.g., a nurse) scans (205), via a handheld scanner, a first side 222 of the double sided label 220 prior to collecting a specimen (e.g., blood) within a specimen tube or container. The first side 222 (having a first barcode or identifier) of the double sided label 220 is scanned after the double sided label 220 is adhered to an outside or exterior of the specimen tube such that the first side 222 of the double sided label 220 faces an interior of the specimen tube and is scannable through an opposite surface of the specimen tube while the specimen tube is empty. In one embodiment, the double sided label is affixed to the container before the specimen collector receives the tube, for example by another group such as the testing lab or material management, or the manufacturer. In addition to scanning the first barcode or identifier (“inner tube ID”), a specimen test order identifier (ID) (e.g., order label) or patient ID (e.g., patient wristband) that is required in a specimen handling process may also be scanned or input into an electronic medical record system. By scanning the specimen test order identifier (ID) and/or a patient ID along with the inner tube ID, the electronic medical record system can link them within a database of the electronic medical record system. Once the specimen is collected in the specimen tube (to which the double sided label 220 is adhered to prior to specimen collection), the inner tube ID will be obscured by the specimen (e.g., blood, urine, etc.) being contained within the specimen tube so that scanning of the inner tube ID will no longer be possible. Obstruction of the inner tube ID allows tracking of the sequence and the timing of events before and after specimen collection. In an exemplary embodiment, if the specimen is collected prior to scanning of the inner tube ID, the specimen would be invalidated by the electronic medical record system and discarded because the inner tube ID would not have been scanned. Therefore, the specimen collection process would have to begin anew. As such, an exemplary method of the present disclosure can prevent mislabeling of specimens by prohibiting “bedside” scanning by the specimen collector or nurse when the tube is full or partially full. Such an exemplary method can ensure procedure follow-through and accountability while not requiring additional resources and significant time.

After successful scanning of the inner tube ID and collection of a specimen, the specimen tube can be sent to a testing laboratory. At the laboratory, the second side 224 (having a second barcode or identifier that is linked to the first barcode/identifier) of the double sided label 220 adhered to the specimen tube is scanned (215) or input along with the specimen test order identifier (ID) and/or patient ID by a representative of the laboratory. It is noted that the second side 224 of the double sided label 220 faces away from an interior of the specimen tube and is visible on an exterior surface of the specimen tube. The electronic record medical system is transmitted (and receives) the second barcode or identifier (“outer tube ID”), the specimen test order ID, and/or patient ID and verifies or correlates (235) that the specimen test order ID/patient ID received from the laboratory (with the outer tube ID) matches the specimen test order ID/patient ID received from the specimen collector (e.g., nurse) (with the inner tube ID), where the electronic medical record system has the inner tube ID and the outer tube ID pre-linked or associated with one another in its database system in advance of initial scanning of the double sided label 220. The electronic record medical system also verifies the sequence of scanning and specimen collection steps as well as the timestamps of the scanning and collection events to ensure they are within the pre-set limits for a specimen to proceed to the laboratory processing step.

If the sequence of specimen collection steps has been altered or the information provided from the specimen testing facility or laboratory does not match with the information provided from a specimen collection facility (e.g., hospital, doctor's office, laboratory, research site, field, etc.), then the electronic medical record system can be configured to reject the specimen and not authorize (245) further testing by the laboratory. In various embodiments, the electronic medical record system may allow for drawing of the specimen to be canceled but the corresponding record may not be deleted from the database of the electronic medical record system. Correspondingly, the electronic medical record system can issue a new order for collection of a new specimen to the specimen collector on behalf of the specimen collection facility to fulfill the provider's original specimen test order.

Otherwise, if the information provided from the testing facility or laboratory matches with the information provided by the specimen collector and any other required information is verified (e.g., the time correlation between the input or scanning of labels/IDs is determined to be acceptable), the electronic medical record system can be configured to approve the specimen for testing so that testing may proceed. Accordingly, the specimen in the specimen tube or container can be tested and results posted to the electronic medical record system.

In various embodiments, a new double sided label for a container is affixed to the tube or container prior to the patient's side or bedside environment. The double sided label may be affixed to the specimen tube/container either by the manufacturer, materials management, the lab, or any other entity prior to the provider's order being issued. Additionally, the information about the time and date a container is collected can be electronically transmitted to a database of an electronic medical record system for retrieval at a later time.

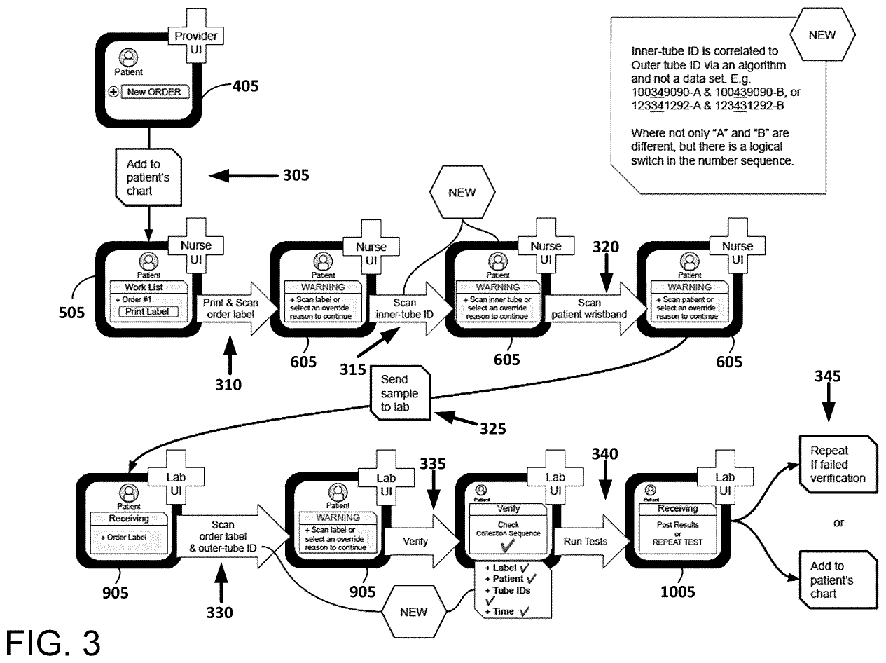

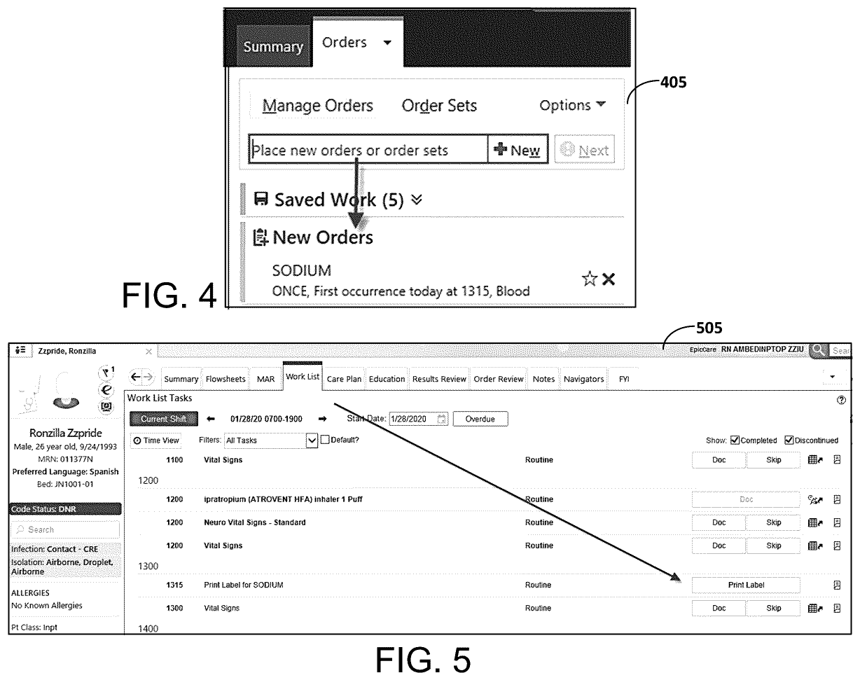

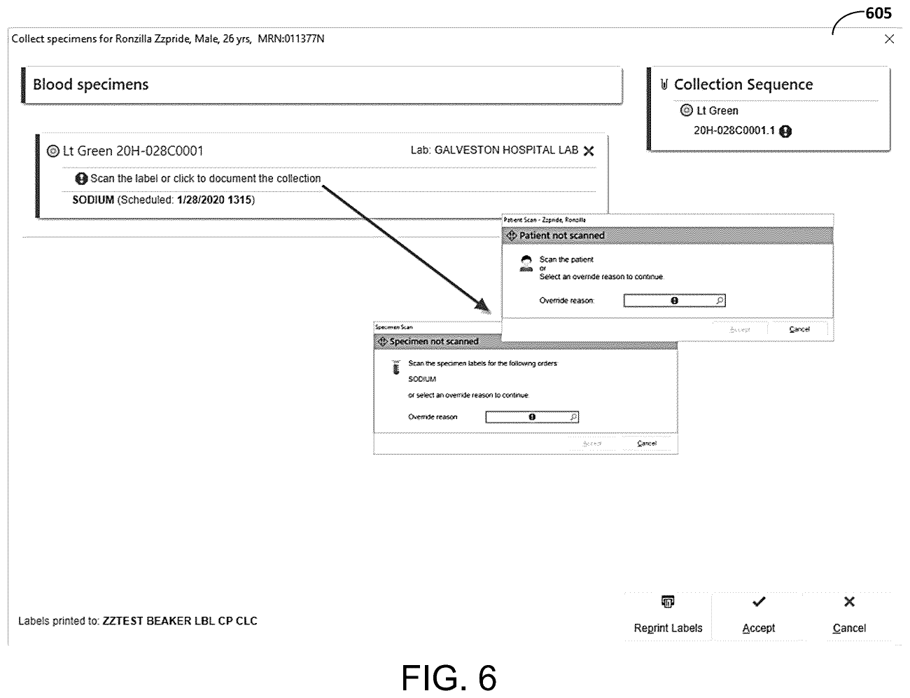

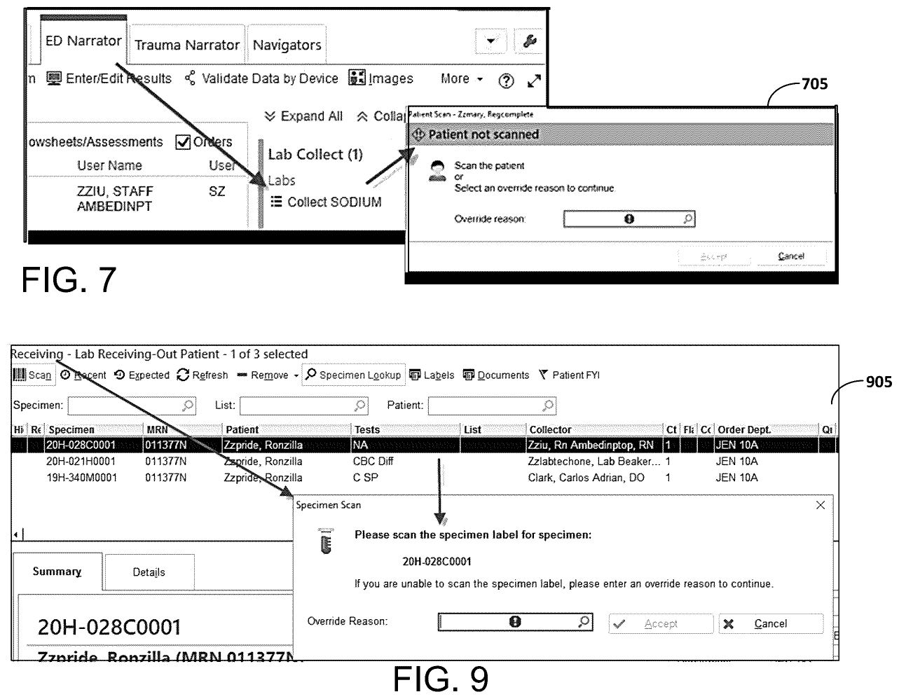

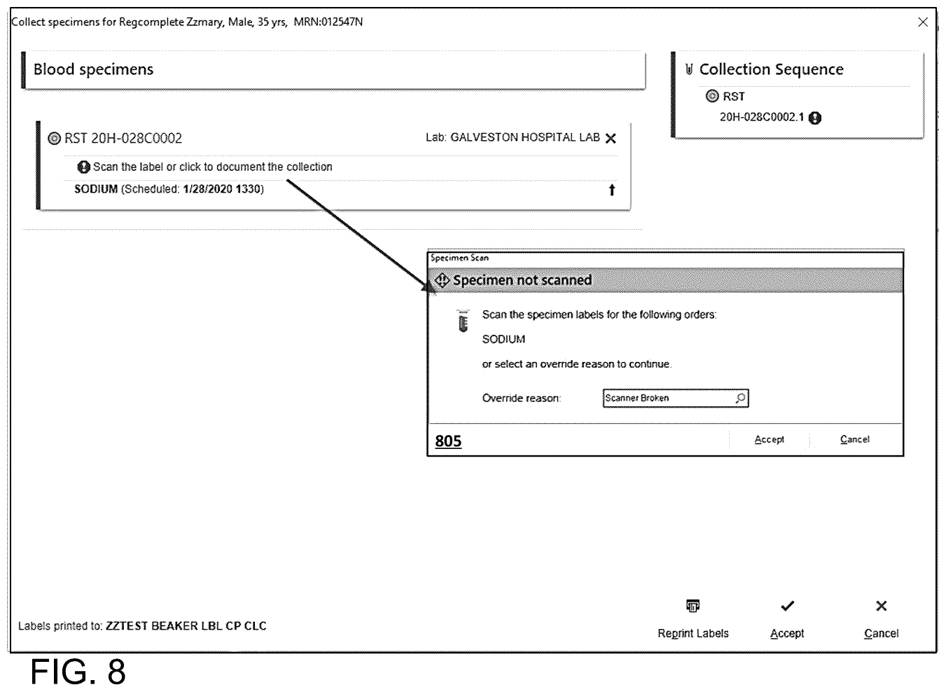

In accordance with various embodiments, the electronic medical record system is integrated with respective interfaces at various medical facilities, such as doctor's offices, lab collection facilities, lab testing facilities, etc. Correspondingly, such interfaces are configured to be operable with and can access a tracking database of the electronic medical record system in various embodiments. Accordingly, FIG. 3 provides an exemplary flow process for interfacing with the electronic medical record system, and FIGS. 4-11 shows exemplary interface screens to the electronic medical record system in accordance with embodiments of the present disclosure. Beginning with FIG. 3, a provider places a specimen test order for a specimen sample. In various embodiments, an electronic medical record system may release, but is not limited to releasing, upcoming orders up to two hours before being due. Accordingly, at a provider user interface, such as a doctor's office or hospital, to the electronic medical record system, a new specimen test order for collecting and testing a specimen can be generated. Correspondingly, FIG. 4 shows a portion of an exemplary user interface screen 405 for placing a new specimen test order for collecting/testing a specimen sample. The generated order can then be added (305) to a patient's records within the electronic medical record system. In accordance with the present disclosure, a specimen collector, such as a nurse, phlebotomist, animal researcher, etc. can view the specimen test order on the nurse's user interface to the electronic medical record system. For example, a nurse, respiratory therapist, phlebotomist, technician, animal researcher, etc. can review specimen test orders from a worklist and be provided the option of issuing a print command for printing, via the label printer, the order label having the specimen test order identifier (ID) and then scanning (310), via a light or image scanner, the specimen test order identifier, as shown in an exemplary user interface screen 505 of FIG. 5. Next, an exemplary user interface can prompt the nurse to scan (315) the inner tube ID for a new specimen tube, as demonstrated by the exemplary interface screen 605 of FIG. 6. Additionally, the user interface can prompt the nurse to scan or input (320) a patient identifier, such as by scanning a barcode on a wristband of the patient, as demonstrated by the exemplary interface screen 605 of FIG. 6. Accordingly, the user interface may display warnings if the scanning operations are not completed, as demonstrated by the exemplary user interface 705 of FIG. 7. After the scanning operations are completed, the identification information for the inner tube ID, patient ID, and/or order ID are transmitted to and recorded by the electronic medical record system and the collected specimen tube or sample is sent (325) to a testing laboratory for analysis. In event that the nurse is unable to perform the scanning steps, the user interface for the nurse may offer the option of selecting or inputting a reason for overriding the scanning procedures, as demonstrated in the exemplary interface screen 805 of FIG. 8. The reasons can then be reviewed at the lab to determine override validity and specimen viability to undergo testing or re-issuing the original order.

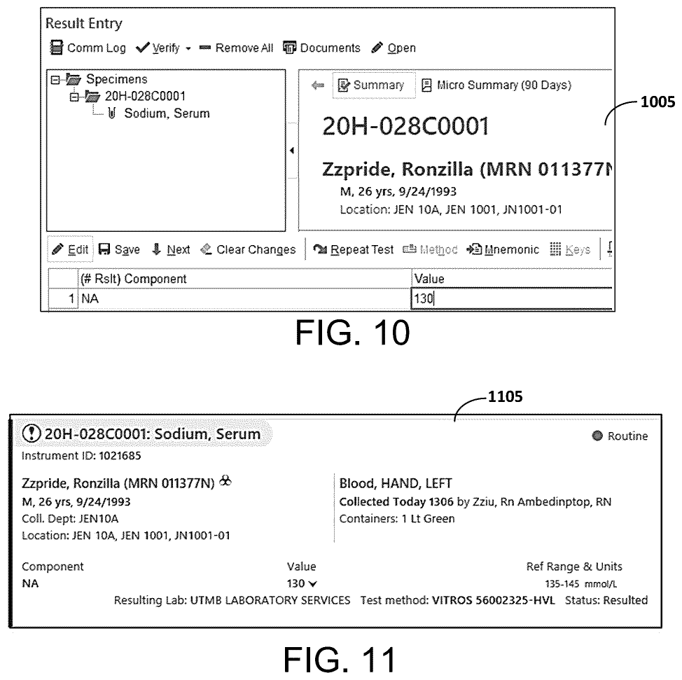

After the specimen testing facility receives the specimen sample, the facility may initiate intake processing of the sample by scanning or inputting (330) the specimen test order label associated with the specimen tube or sample and the outer tube ID 224 located on an exterior surface of the specimen tube, as demonstrated by the exemplary interface screen 905 of FIG. 9 for a specimen testing facility. After verifying (335) that the collected information corresponds or matches with the information stored in the database of the electronic medical record system (e.g., verifying specimen test order label ID, patient ID, inner tube ID, and outer tube ID are correctly linked to one another and/or other requirements are met such as the time requirements for completing the specimen collection and testing process and the specimen scan override validity), testing of the specimen can be authorized, by the electronic medical record system, to be executed (340) or completed by the specimen testing facility. After completion of the test(s), results of the specimen testing can be input into the electronic medical record system, as demonstrated by the exemplary user interface 1005 of FIG. 10. As such, a record 1105 of the test results can be stored on the electronic record system, added to the patient's records (345), and/or made available to a patient's provider (e.g. doctor), as demonstrated by FIG. 11. If the collected information is not able to be verified, however, the collection and testing process will need to be repeated (345) in various embodiments.

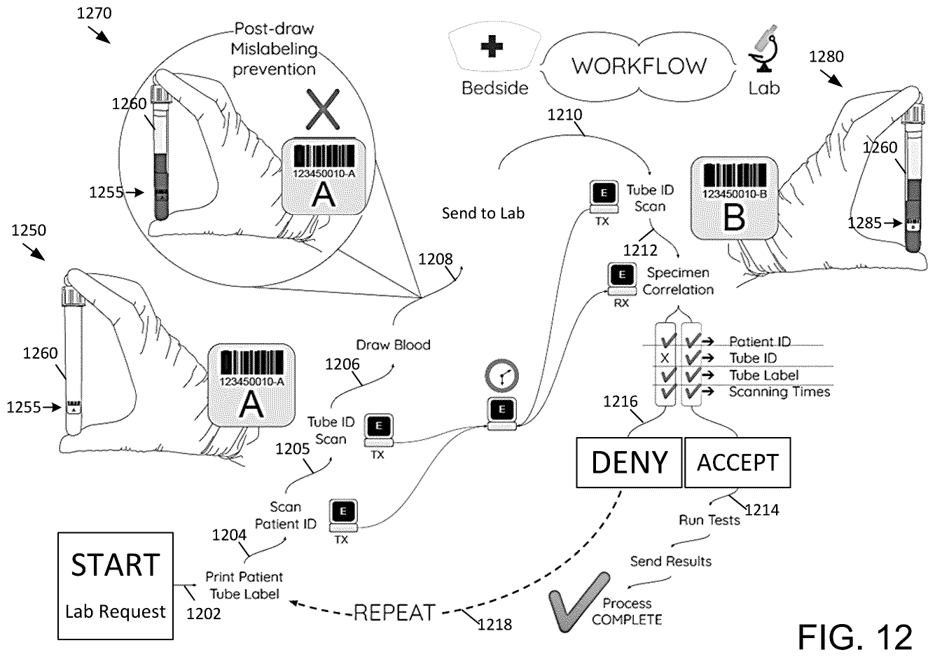

Referring now to FIG. 12, an exemplary workflow process for labeling and collecting a specimen is demonstrated, in accordance with various embodiments of the present disclosure. To begin, a double sided container label is printed (1202) so that the label may be applied to an outer surface of a specimen tube prior to the provider's order being received, in which a sticky or adhesive surface of the inner-side of the label has a first identifier (labeled A in the figure) and the opposite, outer side of the label has a second identifier (labeled B in the figure). Next, the inner-side of the container label having the first identifier is scanned (1205) and associated with patient identification information (that may also be scanned (1204)) and input into an electronic medical record system. As demonstrated by image 1250, the first identifier 1255 (inner tube ID) is visible through a transparent wall of the specimen tube (e.g., a glass tube) and is available to be scanned from an exterior and through the specimen tube when the specimen tube 1260 is empty. Correspondingly, as demonstrated in image 1270, the first identifier (inner tube ID) 1255 is not able to be scanned after the tube 1260 is filled, since the inner tube ID is obscured by the collected sample which prevents post-draw mislabeling of the specimen tube. Accordingly, after collection (1206) of a specimen sample, such as blood, in the specimen tube, the sample is ready for sending (1208) to a specimen testing facility.

At the specimen testing facility, the second identifier (outer tube ID) on the outer side (non-adhesive) of the double sided label 220 is scanned (1210). Correspondingly, as demonstrated in image 1280, the second identifier (outer tube ID) 1285 is able to be scanned after the tube 1260 is filled, since the outer tube ID is visible on an exterior of the specimen tube 1260. The scanner outer tube ID is transmitted along with the specimen test order ID to the electronic medical record system, such that the system can verify (1212) that the outer tube ID (second identifier) is associated with the specimen test order ID in the database of the electronic medical record system by confirming that an inner tube ID (first identifier) associated with the outer tube ID is also associated with the specimen test order ID. Accordingly, a variety of checks can be performed such as verifying that a patient ID associated with the outer tube ID matches with a patient ID associated with the inner tube ID, verifying that the outer tube ID is linked to the inner tube ID, verifying that a specimen test order ID associated with the outer tube ID matches a specimen test order ID associated with the inner tube ID, etc. If the one or more verification checks are successfully completed, then the test of the specimen sample can be authorized (1214) to be performed by the electronic medical record system, the tests can be performed, and/or the results returned to the electronic medical record system. Otherwise, the system will not allow processing (1216) of the specimen sample if the verification checks are not successfully completed and the process may be repeated (1218) in some embodiments.

In accordance with embodiments of the present disclosure, an exemplary double sided label contains bar codes or other identifiers that are capable of being scanned or imaged using an scanning apparatus or reader. As such, a variety of scanning or imaging technologies can be utilized in various embodiments in accordance with the present disclosure. For example, in some embodiments, an optical light scanner can read printed barcode(s) of the double sided label, wherein the scanner has a sensor that detects light or laser patterns that is transmitted by the scanner/reader and reflected from the barcode. Alternatively, image scanners, CCD/LED (charge coupled device / light emitting diode) scanners, etc. can be used to read and decode a barcode or other type of identifier pattern, for example, computer vision. Correspondingly, a variety of barcodes may be utilized in various embodiments, such as linear barcodes, data matrix barcodes, quick response (QR) codes, etc. Further, in some embodiments, barcodes may be oriented in a horizontal or vertical direction based on the shape of the specimen tube or vessel to which the double sided label is to be applied. Accordingly, if the label is large in relation a circumference of the tube/vessel, the label may be oriented in a vertical direction to make scanning of the label on a curved surface of the tube/vessel easier to read by a light or image scanner.

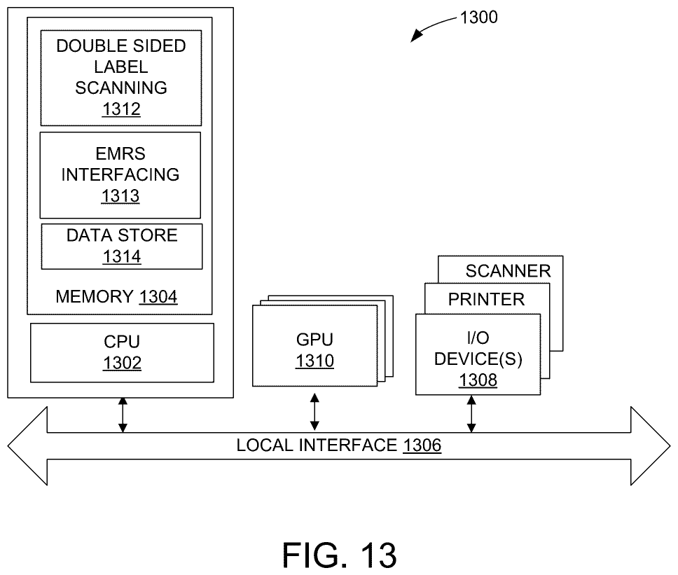

FIG. 13 depicts a schematic block diagram of a computing device 1300 that can be used to implement various embodiments of the present disclosure. An exemplary computing device 1300 includes at least one processor circuit, for example, having a processor 1302 and a memory 1304, both of which are coupled to a local interface 1306, and one or more input and output (I/O) devices 1308, such as an optical light or image scanner. The local interface 1306 may comprise, for example, a data bus with an accompanying address/control bus or other bus structure as can be appreciated. The computing device 1300 further includes Graphical Processing Unit(s) (GPU) 1310 that are coupled to the local interface 1306 and may utilize memory 1304 and/or may have its own dedicated memory. In various embodiments, the CPU and/or GPU(s) can perform various operations such as image enhancement, image/video processing, recognition (e.g., text recognition, object recognition, feature recognition, etc.), image classification, and any of the various operations described herein.

Stored in the memory 1304 are data, instructions, commands, and several components that are executable by the processor 1302. In particular, stored in the memory 1304 and executable by the processor 1302 are code 1312 for scanning the double sided label (“double sided label scanning code”) and code for 1313 interfacing with the electronic medical record system (“EMRS interfacing code”). Also stored in the memory 1304 may be a data store 1314 and other data. In addition, an operating system may be stored in the memory 1304 and executable by the processor 1302. The I/O devices 1308 may include input devices, for example but not limited to, cameras, barcode/image scanners, a keyboard, touchscreen, mouse, etc. Furthermore, the I/O devices 1308 may also include output devices, for example but not limited to a label printer, speaker, earbuds, audio output port, display, Bluetooth output module, etc.

It should be emphasized that the above-described embodiments are merely possible examples of implementations, merely set forth for a clear understanding of the principles of the present disclosure. Many variations and modifications may be made to the above-described embodiment(s) without departing substantially from the principles of the present disclosure. All such modifications and variations are intended to be included herein within the scope of this disclosure.

Therefore, at least the following is claimed:

1. A method comprising:
linking, by a computing device, a pair of identification numbers with one another within a specimen test order for a subject in an electronic medical record system in advance of the pair of identification numbers being printed on a double sided container label;
wherein a first one of the pair of identification numbers is represented by a first machine readable identifier on a first side of the double sided container label, wherein a second one of the pair of identification numbers is represented by a second machine readable identifier on a second side of the double sided container label, wherein the first side of the double sided container label has an adhesive for attaching to a specimen container;
receiving, by the computing device via a scanner device, the first machine readable identifier on the first side of the container label before a specimen is collected in the specimen container;
transmitting, by the computing device, the first identification number represented by the scanned first machine readable identifier to the electronic medical record system, wherein the electronic medical record system verifies that the first identification number is associated with the specimen test order for the subject;
receiving, by the computing device via a scanner device, the second machine readable identifier on the second side of the container label after the specimen is collected in the specimen container;
transmitting, by the computing device, the second identification number represented by the scanned second machine readable identifier to the electronic medical record system, wherein the electronic medical record system verifies that the second identification number is associated with the specimen test order for the subject and/or the first machine readable identifier; and
authorizing, by the computing device, the specimen to be tested by the specimen testing facility after verifying that the second identification number is associated with the specimen test order and/or the first machine readable identifier for the subject.

2. The method of claim 1, wherein a portion of the first identification number is the same as a corresponding portion of the second identification number and a different portion of the first identification number is different than a corresponding portion of the second identification number.

3. The method of claim 1, wherein the first machine readable identifier on the first side of the double sided label is scanned after the first side of the double sided label is adhered to an exterior side of the specimen container such that the first side of the double sided label faces an interior of the specimen container.

4. The method of claim 1, wherein the specimen container is transparent.

5. The method of claim 1, wherein the first machine readable identifier on the first side of the double sided label is scanned through the specimen container after the first side of the double sided label is adhered to an exterior side of the specimen container such that the first side of the double sided label faces an interior of the specimen container.

6. The method of claim 1, further comprising reading, by the computing device via the scanning device, a subject identifier; and transmitting, by the computing device, the subject identifier to the electronic medical record system, wherein the electronic medical record system verifies that the subject identifier is associated with the scanned first identification number.

7. The method of claim 1, further comprising reading, by the computing device via the scanning device, a specimen test order identifier; and transmitting, by the computing device, the specimen test order identifier to the electronic medical record system, wherein the electronic medical record system verifies that the specimen test order identifier is associated with the scanned first identification number.

8. The method of claim 1, further comprising posting, by the computing device, specimen testing results to the electronic medical record system.

9. The method of claim 1, wherein the subject is a mammal.

10. The method of claim 9, wherein the mammal is a human.

11. A system comprising:
a computing device having a processor and memory storage, wherein the memory storage stores executable-instructions, that when executed by the processor, causes the computing device to perform various operations, the various operations including:
linking a pair of identification numbers with one another within a specimen test order for a patient in an electronic medical record system in advance of the pair of identification numbers being printed on a double sided container label;
wherein a first one of the pair of identification numbers is represented by a first machine readable identifier on a first side of the double sided container label, wherein a second one of the pair of identification numbers is represented by a second machine readable identifier on a second side of the double sided container label, wherein the first side of the double sided container label has an adhesive for attaching to a specimen container;
receiving, via a scanner device, the first machine readable identifier on the first side of the container label before a specimen is collected in the specimen container;
transmitting the first identification number represented by the scanned first machine readable identifier to the electronic medical record system, wherein the electronic medical record system verifies that the first identification number is associated with the specimen test order for the patient;
receiving, via a scanner device at a specimen testing facility, the second machine readable identifier on the second side of the container label after the specimen is collected in the specimen container;
transmitting the second identification number represented by the scanned second machine readable identifier to the electronic medical record system, wherein the electronic medical record system verifies that the second identification number is associated with the specimen test order for the patient; and
authorizing the specimen to be tested by the specimen testing facility after verifying that the second identification number is associated with the specimen test order for the patient.

12. The system of claim 11, wherein a portion of the first identification number is the same as a corresponding portion of the second identification number and a different portion of the first identification number is different than a corresponding portion of the second identification number.

13. The system of claim 11, wherein the specimen container is transparent.

14. The system of claim 11, wherein the operations further comprises reading, via the scanning device, a patient identifier; and transmitting the patient identifier to the electronic medical record system, wherein the electronic medical record system verifies that the patient identifier is associated with the scanned first identification number.

15. The system of claim 11, wherein the operations further comprise reading, via the scanning device, a specimen test order identifier; and transmitting the specimen test order identifier to the electronic medical record system, wherein the electronic medical record system verifies that the specimen test order identifier is associated with the scanned first identification number.

16. The system of claim 11, further comprising the label printer.

17. The system of claim 11, further comprising the scanner device at a specimen collection facility or at a specimen testing facility.

18. A computer readable storage medium having instructions stored thereon that in response to execution by a computing device, cause the computing device to perform operations comprising:
linking a pair of identification numbers with one another within a specimen test order for a patient in an electronic medical record system in advance of the pair of identification numbers being printed on a double sided container label;
issuing a print command to a label printer for printing the double sided container label, wherein a first one of the pair of identification numbers is represented by a first machine readable identifier on a first side of the double sided container label, wherein a second one of the pair of identification numbers is represented by a second machine readable identifier on a second side of the double sided container label, wherein the first side of the double sided container label has an adhesive for attaching to a specimen container;
receiving, via a scanner device, the first machine readable identifier on the first side of the container label before a specimen is collected in the specimen container;
transmitting the first identification number represented by the scanned first machine readable identifier to the electronic medical record system, wherein the electronic medical record system verifies that the first identification number is associated with the specimen test order for the patient;
receiving, via a scanner device at a specimen testing facility, the second machine readable identifier on the second side of the container label after the specimen is collected in the specimen container;
transmitting the second identification number represented by the scanned second machine readable identifier to the electronic medical record system, wherein the electronic medical record system verifies that the second identification number is associated with the specimen test order for the patient; and
authorizing the specimen to be tested by the specimen testing facility after verifying that the second identification number is associated with the specimen test order for the patient.

19. The computer readable storage medium of claim 18, wherein a portion of the first identification number is the same as a corresponding portion of the second identification number and a different portion of the first identification number is different than a corresponding portion of the second identification number.

20. The computer readable storage medium of claim 18, wherein the operations further comprise reading, via the scanning device, a patient identifier; and transmitting the patient identifier to the electronic medical record system, wherein the electronic medical record system verifies that the patient identifier is associated with the scanned first identification number.

21. The computer readable storage medium of claim 18, wherein the operations further comprises reading, via the scanning device, a specimen test order identifier; and
transmitting the specimen test order identifier to the electronic medical record system, wherein the electronic medical record system verifies that the specimen test order identifier is associated with the scanned first identification number.
